(19) United States
(12) Patent Application Publication
(10) Pub. No.: US 2020/0390739 A1
(43) Pub. Date: Dec. 17, 2020

(54) HEMP-BASED NUTRACEUTICAL

(71) Applicant: Gaia Herbs, Brevard, NC (US)

(72) Inventors: Jeremy Stewart, Brevard, NC (US); Logan Breiner, Brevard, NC (US); Greg Masters, Brevard, NC (US)

(21) Appl. No.: 16/902,673

(22) Filed: Jun. 16, 2020

Related U.S. Application Data
(60) Provisional application No. 62/862,262, filed on Jun. 17, 2019.

(51) Int. Cl. A61K 31/352; A61K 31/05; A61K 47/46; A61K 47/12; A61K 9/48

(52) U.S. Cl. CPC A61K 31/352; A61K 31/05; A61K 2236/333; A61K 47/12; A61K 9/4816; A61K 47/46

(57) ABSTRACT

The present invention relates to a process for providing hemp extracts in a vegetable gelatin, HPMC, or any other cellulose derivative capsule. The process includes extracting a hemp plant material with an aqueous alcohol mixture to provide an aqueous alcoholic hemp extract. The aqueous alcoholic hemp extract is concentrated with an emulsifier (e.g., lecithin) and diluted with a glycerol (e.g., glycerin) while maintaining a carboxylation:decarboxylation ratio equal to or greater than about 2:1 for the plurality of cannabinoids present within the hemp extract. Alcohol and water is removed from the mixture to provide a semi-soft form hemp extract. The herb extract is then encapsulated in a vegetable gelatin, HPMC, or any other cellulose derivative capsule.

### RELATED APPLICATION

The following application claims priority to U.S. Provisional No. 62/862,262 filed Jun. 17, 2019, the disclosure of which is incorporated by reference in its entirety.

### FIELD OF THE INVENTION

The present invention relates to a process of providing hemp extracts in cellulose derivative capsules, and more particularly, a semi-soft liquid form of hemp extracts in vegetable gelatin, hydroxypropyl methylcellulose (“HPMC”), or any other cellulose derivative capsules.

### BACKGROUND OF THE INVENTION

Cannabis plants contain more than 400 chemicals and approximately 80 cannabinoids, including tetrahydrocannabinol (THC), cannabidiol (CBD), cannabinol (CBN), tetrahydrocannabivarin (THCV) and cannabigerol (CBG). The primary psychoactive constituent of cannabis is THC, which may be used for treating a wide range of medical conditions, including glaucoma, AIDS wasting, neuropathic pain, treatment of spasticity associated with multiple sclerosis, fibromyalgia and chemotherapy-induced nausea. THC is also found to be effective in the treatment of allergies, inflammation, infection, epilepsy, depression, migraine, bipolar disorders, anxiety disorder, drug dependency and drug withdrawal syndromes. However, the adoption of THC as a pharmaceutical drug is impeded by its psychotropic side effects.

Other cannabinoids also exhibit certain pharmacological activity and provide various health benefits. For example, cannabidiol (CBD) is a potent antioxidant and anti-inflammatory compound known to provide protection against acute and chronic neurodegeneration; cannabigerol (CBG), found in high concentrations in hemp, which acts as a high affinity a2-adrenergic receptor agonist, moderate affinity 5-HT1A receptor antagonist and low affinity CB1 receptor antagonist, and possibly has anti-depressant activity; and cannabichromene (CBC), which possesses anti-inflammatory, anti-fungal and anti-viral properties.

Hemp plants are a class of Cannabis varieties that contain 0.3% or less THC content (by dry weight) according to the Agricultural Act of 2018 and are thus desirable for obtaining extracts of cannabinoids with minimal THC content. While the decarboxylated forms of these cannabinoids may still produce some psychoactive side effects, it has been found that retaining the cannabinoids in their carboxylated form reduces and/or eliminates their psychoactivity. Thus, it is desirable to extract the cannabinoids from hemp plant materials while maintaining them in their carboxylated forms.

Moreover, the delivery system for such hemp extracts plays a significant role in the way and the extent to which the compounds are metabolized. It has also been suggested that a liquid delivery system is a superior method of administering therapeutic remedies.

Therefore, there remains a need for capsules containing a semi-soft liquid form hemp extract from hemp plant materials and methods of preparing thereof, wherein the cannabinoids within the hemp extract are preserved in a predominately carboxylated form.

### SUMMARY OF THE INVENTION

Consumer demand for hemp-based products is increasing rapidly today. This invention specifies the procedures of providing a semi-soft liquid form hemp extract in a sealed cellulose derivative capsule such as vegetable gelatin or HPMC. This technology demonstrates the unique methods and processes utilized in extraction that enable the liquid hemp extract and vegetable gelatin, HPMC, or any other cellulose derivative capsule to remain stable without degradation once the liquid is filled into the capsule.

In one aspect, the present invention relates to a semi-soft hemp extract encapsulated in a cellulose derivative capsule. Another aspect of the present invention relates to a process for providing a semi-soft liquid-form hemp extract in a vegetable gelatin, HPMC, or any other cellulose derivative capsule. In one embodiment, the process includes extracting a hemp plant material with an alcohol or aqueous/alcohol to provide an aqueous alcoholic hemp extract containing a plurality of cannabinoids. This aqueous alcoholic hemp extract is transferred to a rotary evaporation or other condensing equipment to evaporate water and alcohol content and further concentrate the extract. An emulsifier (e.g., lecithin) is added to the extract while the water and alcohol evaporate in the condensing equipment. A glycol (e.g., glycerin) is added to form a glycerin:lecithin hemp extract, which is maintained in solution or dispersed in the alcohol mixture. The alcohol and water are removed from the glycerin:lecithin hemp extract to provide a semi-soft form hemp extract, which is then encapsulated in vegetable gelatin, HPMC, or any other cellulose derivative capsule.

It is an object of the present methods to prevent decarboxylation of the cannabinoids during the extraction process. The extraction method disclosed herein prevents decarboxylation of the cannabinoids. The cannabinoids within the hemp extract may have a carboxylation:decarboxylation ratio equal to or greater than about 2:1.

### DETAILED DESCRIPTION OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items.

The term “about,” as used herein when referring to a measurable value such as an amount of a compound, dose, time, temperature, and the like, is meant to encompass variations of 20%, 10%, 5%, 1%, 0.5%, or even 0.1% of the specified amount. Unless otherwise defined, all terms, including technical and scientific terms used in the description, have the same meaning as commonly understood by one of ordinary skill in the art to which this invention belongs.

As used herein, the terms “comprise,” “comprises,” “comprising,” “include,” “includes” and “including” specify the presence of stated features, integers, steps, operations, elements, and/or components, but do not preclude the presence or addition of one or more other features, integers, steps, operations, elements, components, and/or groups thereof.

As used herein, the term “consists essentially of” (and grammatical variants thereof), as applied to the compositions and methods of the present invention, means that the compositions/methods may contain additional components so long as the additional components do not materially alter the composition/method. The term “materially alter,” as applied to a composition/method, refers to an increase or decrease in the effectiveness of the composition/method of at least about 20% or more.

All patents, patent applications and publications referred to herein are incorporated by reference in their entirety. In case of a conflict in terminology, the present specification is controlling.

As discussed above, a hemp plant material is provided. The hemp material is then extracted with an aqueous alcohol in different concentrations to provide an aqueous alcoholic hemp extract. Suitable alcohols include C1 to C3 alcohols like ethanol. Preferably, ethanol is used. The alcohol can be a co-solvent mixture such as a mixture of alcohol and water. For example, the alcohol mixture may be an ethanol solution ranging from about 70% w/v to about 90% w/v. In a preferred embodiment, a 70% w/v ethanol solution is used to extract the hemp material. During extraction, the hemp material is preferably percolated or macerated to facilitate extraction.

The resulting aqueous alcoholic hemp extract is comprised of a plurality of cannabinoids. Examples of cannabinoids that may be found in the extract include cannabinol, cannabinolic acid, cannabidiol, cannabidiolic acid, canabidivarin, cannabidivarinic acid, cannabichromene, cannabichromenic acid, cannabidiolic acid, cannabidivarin, cannabigerol, cannabigerolic acid, and cannabigerivarin. The carboxylated forms of the appropriate cannabinoids within the hemp extract are preferably present in a higher ratio than their decarboxylated counterparts. In one embodiment, the cannabinoids within the hemp extract are preserved in a predominately carboxylated form, wherein the carboxylation:decarboxylation ratio is equal to or greater than about 2:1. For example, the carboxylation:decaboxylation ratio for each cannabinoid may be equal to about 3:1. The carboxylation ratios of the cannabinoids may be further modified; for example, by using heat or light.

The aqueous alcoholic hemp extract may be monitored for its bio-activity. As used herein, bio-activity is defined as qualitative and quantitative measurement of the marker compounds.

After extraction, an emulsifier is added to the aqueous alcoholic hemp extract and the solvent is removed by distillation to form an emulsion:hemp extract, which is then diluted with a glycol to adjust the bio-activity required to complete the standardization of the formulation and form a glycol emulsion for the extract. Glycol is preferably added after addition of the emulsifier to preserve the carboxylation:decarboxylation ratio of the extract.

In one preferred embodiment, the emulsifier is lecithin, which also serves as a lipid-binding excipient. Examples of suitable lecithin sources include corn lecithin, cottonseed oil lecithin, egg-yolk lecithin, rapeseed lecithin, soybean lecithin and sunflower lecithin. A preferred glycol is glycerin. However, other suitable examples may include polyols such as polypropylene glycol.

Preferably, the amount of glycerin added to the hemp extract is not equal to the amount of lecithin added. In one embodiment, the glycerin:lecithin ratio is about 3:1. In a more preferred embodiment, the glycerin:lecithin ratio is about 1:3. Adding glycerin and lecithin at a 1:1 ratio results in an emulsion with an unfavorably high viscosity that interferes with the encapsulation process, and therefore is disfavored. Without wishing to be bound by theory, it is believed that a 1:1 ratio results in an unstable emulsion for the hemp extract possibly due to interactions between lecithin and glycerin or air trapped within the emulsion.

Typically about 15 to about 35 percent by weight of glycerin and about 65 to about 85 percent by weight of lecithin is contained in the finished product. The mixture of aqueous alcoholic hemp extract, lecithin and glycerin is condensed or concentrated, using any one of the various condensation techniques known to those skilled in the art. For example, rotary evaporation under reduced pressure in a warm water bath at a temperature of from about 55° to 85° C. can be used.

Preservatives may be added to maintain the carboxylation:decarboxylation ratio for the plurality cannabinoids present in the hemp extract. For example, the preservative may be an organic acid adapted to maintain the carboxyl groups of the cannabinoids in a protonated state. Examples of suitable organic acids include citric acid or any other non-volatile organic acid. Another example of a preservative may be an antioxidant preventing oxidative degradation of the cannabinoids. Examples of suitable antioxidants include rosemary extract, sage extract, clove-bud, mixed-tocopherols, or green tea extract.

The hemp extract is then encapsulated in a cellulose derivative capsule. Suitable cellulose derivatives include, but are not limited to, vegetable gelatin and hydroxylalkyl celluloses including methylcellulose, carboxymethylcellulose, hydroxypropylcellulose, and the preferred hydroxypropyl methylcellulose (“HPMC”). Suitable capsules are available from many sources, and sizes from “00” to “3” are preferably used. Suitable excipients may be added to the extract prior to encapsulation and may include vegetable oils, waxes, lecithin, fats, semi-solid and liquid polyols and the like. Preferably, no excipients other than lecithin are needed. Suitable encapsulation equipment is available from market suppliers such as Shionogi. Air can be eliminated from the capsules using an inert gas such as nitrogen.

Other herbal materials may be included in combination with the hemp extract within the cellulose derivative capsule. The herbal material is preferably in the form of whole leaf, stem, stalk, root and the like, and is ground or cut prior to treatment. The herbal materials can be organic, cultivated, or wild. Suitable herbal materials include, but are not limited to, kava root, echinacea, St. John's wort, valerian root, milk thistle seed, Siberian ginseng, nettle leaf, ginkgo, gotu kola, ginkgo/gotu kola supreme, astragalus, goldenseal, dong quai, ginseng, St. John's wort supreme, echinacea/goldenseal supreme, bilberry, green tea, hawthorn, ginger, turmeric, black cohosh, cats claw, chamomile, dandelion, chaste tree berry, feverfew, garlic, horse chestnut, licorice, eyebright, yohimbe, astragalus supreme, valerian poppy supreme, and serenity elixir.

The following examples are merely illustrative of the invention and are not limiting thereon.

### EXAMPLES

### Preparation of Hemp Extracts and Encapsulation

An aqueous alcoholic hemp extract is obtained from aerial portions of Cannabis sativa, ideally flowers, with a mixture of ethyl alcohol and water having a concentration of about 70% w/v. The aqueous alcoholic hemp extract is filtered through a fine screen (100 mesh).

In one embodiment, 50 kg of hemp flower is extracted with 500 L of 70% aqueous ethanol. The filtered extract is transferred to a concentrator, and the aqueous ethanol is removed by vacuum distillation. During concentration, 5 kg lecithin and 1.7 kg glycerin are injected into the concentrator, and the process is complete after the extract has a moisture content of less than or equal to about 25%.

The concentrated extract is removed from the concentrator. A sample is sent for testing and the extract yield is adjusted with lecithin/glycerin (3:1) in accordance with the test results to a finished cannabinoid concentration of 100-115 mg/g.

The extract is encapsulated in a size “1” HPMC vegetable capsule and sealed with the same HPMC material. Each capsule holds 0.56-0.58 mL of hemp extract.

Although the present approach has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present approach.

Yhat which is claimed is:

1. A process for providing hemp extracts in cellulose derivative capsules, the process comprising the steps of:
(a) extracting a hemp plant material with an aqueous alcohol mixture to provide an aqueous alcoholic hemp extract having a plurality of cannabinoids, wherein the cannabinoids have a carboxylation:decarboxylation ratio equal to or greater than about 2:1;
(b) concentrating the aqueous alcoholic hemp extract with an emulsifier to provide an emulsified hemp extract, wherein the cannabinoids have a carboxylation:decarboxylation ratio equal to or greater than about 2:1;
(c) adding a glycol to the emulsified hemp extract to provide anemulsified glycol hemp extract maintained in solution or dispersed in the aqueous alcohol mixture;
(d) removing alcohol and water from theemulsfied glycol hemp extract to provide a semi-soft form hemp extract; and
(e) encapsulating the semi-soft form hemp extract in a cellulose derivative capsule.

2. The process of claim 1, wherein the plurality of cannabinoids comprises cannabinol, cannabinolic acid, cannabidiol, cannabidiolic acid, canabidivarin, cannabidivarinic acid, cannabichromene, cannabichromenic acid, cannabidiolic acid, cannabidivarin, cannabigerol, cannabigerolic acid, cannabigerivarin and combinations thereof.

3. The process of claim 1, wherein the carboxylation:decarboxylation ratio is about 3:1.

4. The process of claim 1 further including adding a preservative to maintain the carboxylation:decarboxylation ratio.

5. The process of claim 4, wherein the preservative is an organic acid for maintaining carboxyl groups of the cannabinoids in a protonated state.

6. The process of claim 5, wherein the organic acid is citric acid.

7. The process of claim 5, wherein the preservative is an antioxidant for preventing oxidation of the cannabinoids.

8. The process of claim 7, wherein the antioxidant is rosemary extract.

9. The process of claim 1, wherein the semi-soft form extract has a moisture content of less than about 10% by weight.

10. The process of claim 1, wherein the semi-soft form extract has a moisture content of less than about 5% by weight.

11. The process of claim 1, wherein bio-activity of the aqueous alcoholic hemp extract is measured after extracting from the hemp plant material.

12. The process of claim 1, wherein bio-activity of the semi-soft form hemp extract is measured before encapsulating the semi-soft hemp extract into the cellulose derivative capsule.

13. The process of claim 1 further including eliminating air in the cellulose derivative capsule by contacting the capsule with an inert gas.

14. The process of claim 13, wherein the inert gas is nitrogen.

15. A process for providing hemp extracts in cellulose derivative capsules, the process comprising the steps of:
(a) extracting a hemp plant material with an aqueous alcohol mixture to provide an aqueous alcoholic hemp extract having a plurality of cannabinoids, wherein the cannabinoids have a carboxylation:decarboxylation ratio equal to or greater than about 2:1;
(b) concentrating the aqueous alcoholic hemp extract with a lecithin to provide a lecithin hemp extract, wherein the cannabinoids have a carboxylation:decarboxylation ratio equal to or greater than about 2:1;
(c) adding glycerin to the lecithin hemp extract to provide a glycerin:lecithin hemp extract maintained in solution or dispersed in the aqueous alcohol mixture;
(d) removing alcohol and water from the glycerin:lecithin hemp extract to provide a semi-soft form hemp extract; and
(e) encapsulating the semi-soft form hemp extract in a cellulose derivative capsule.

16. The process of claim 15, wherein the glycerin:lecithin hemp extract has a glycerin:lecithin ratio that is not equal to 1:1.

17. The process of claim 16, wherein the glycerin:lecithin ratio is about 1:3.

18. A cellulose derivative capsule comprising a semi-soft hemp extract having a plurality of cannabinoids, wherein the cannabinoids have a carboxylation:decarboxylation ratio equal to or greater than about 2:1.

19. The cellulose derivative capsule of claim 18, wherein the plurality of cannabinoids comprises cannabinol, cannabinolic acid, cannabidiol, cannabidiolic acid, canabidivarin, cannabidivarinic acid, cannabichromene, cannabichromenic acid, cannabidiolic acid, cannabidivarin, cannabigerol, cannabigerolic acid, cannabigerivarin and combinations thereof.

20. The cellulose derivative capsule of claim 18, wherein the carboxylation:decarboxylation ratio is about 3:1.

21. The cellulose derivative capsule of claim 18 further including a preservative to maintain the carboxylation: decarboxylation ratio.

22. The cellulose derivative capsule of claim 21, wherein the preservative is an organic acid for maintaining carboxyl groups of the cannabinoids in a protonated state.

23. The cellulose derivative capsule of claim 22, wherein the organic acid is citric acid.

24. The cellulose derivative capsule of claim 22, wherein the preservative is an antioxidant for preventing oxidation of the cannabinoids.

25. The cellulose derivative capsule of claim 24, wherein the antioxidant is rosemary extract.

26. The cellulose derivative capsule of claim 18, wherein the semi-soft hemp extract has a moisture content of less than about 10% by weight.

27. The cellulose derivative capsule of claim 26, wherein the semi-soft hemp extract has a moisture content of less than about 5% by weight.
